(19) United States
(12) Patent Application Publication
(10) Pub. No.: US 2007/0216865 A1
(43) Pub. Date: Sep. 20, 2007

(54) Process and Device for Apportioning Therapeutic Vision Stimuli

(75) Inventors: Bernhard Sabel, Berlin (DE); Dorothee Schlueter, Sulzetal (DE); Sigrid Kenkel, Boca Raton, FL (US)

Correspondence Address: BROMBERG & SUNSTEIN LLP, 125 SUMMER STREET, BOSTON, MA, 02110-1618, US

(73) Assignee: NOVAVISION, INC., Boca Raton, FL (US)

(21) Appl. No.: 11/689,230

(22) Filed: Mar. 21, 2007

Related U.S. Application Data
(63) Continuation-in-part of application No. 10/503,869, filed on May 18, 2005.
(60) Provisional application No. 60/784,235, filed on Mar. 21, 2006.

(51) Int. Cl. A61B 3/00

(52) U.S. Cl. 351/203

(57) ABSTRACT

A method and device for treating the visual system of a human is provided. The method comprises situating the human in proximity to a computer-actuated light emitting array that has a set of individually actuable elements. A campimetric representation of the visual field is used to select a stimulus distribution. An actuable element subset is selected from the set of individually actuable elements based on the stimulus distribution. The subset of elements is actuated to emit a light stimulus that is directed to a specified region of the human's visual field. The stimulus distribution may be biased toward the central visual field or based on apportioning among specified zones. The distribution may also be based on a transition zone.

### CROSS REFERENCE TO RELATED APPLICATIONS

This application claims priority from U.S. Provisional Patent Application Ser. No. 60/784,235, filed Mar. 21, 2006, and is a continuation-in-part of U.S. patent application Ser. No. 10/503,869, filed May 18, 2005 in the United States, which claims priority from PCT Patent Application No. PCT/EP02/01339, which was filed in English on Feb. 8, 2002; all of these applications are hereby incorporated by reference in their entirety.

### TECHNICAL FIELD

The present invention relates to focused vision therapy and, in particular, to selectively apportioning light stimulation to different areas of a patient's visual field.

### BACKGROUND

Stimulating the vision system of human subjects with vision impairment may improve their visual performance. For example, as documented in U.S. Pat. No. 6,464,356, and US Published Patent Application No. 2005/0213033, which are hereby incorporated by reference herein in their entirety, presenting visual stimuli to areas of a human's visual system may allow improvement in the user's vision. NovaVision, of Boca Raton, Fla., produces VRT™ (Visual Restoration Therapy) devices for effecting optical stimulation of defined locations of a patient's retina. During a course of VRT, a finite number of stimulation events are available. Therefore, these stimulation events should be judiciously directed to the particular visual field regions for which treatment is desired.

VRT may be used to treat neurological deficits of the visual system of a patient. Such deficits may result from retinal damage, damage to the optic nerve, damage to the visual cortex, such as may occur due to stroke or traumatic brain injury. For example, age related macular degeneration (AMD) may be treated with VRT.

### SUMMARY OF THE INVENTION

In accordance with an illustrative embodiment of the invention, there is a method for treating the visual system of a human. The method comprises situating the human in proximity to a computer-actuated light emitting array that has a set of individually actuable elements. A campimetric representation of the visual field is used to select a stimulus distribution that is biased toward the central visual field. An actuable element subset is selected from the set of individually actuable elements based on the stimulus distribution. The subset of elements is actuated to emit a light stimulus that is directed to a specified region of the human's visual field.

Various related embodiments are provided including optional or additional features. For example, a fixation stimulus may be presented to the human. The steps of selecting and actuating the light stimulus may be repeated for a given number of cycles. A further step of recording the human's response to the stimuli may be included. The steps of selecting the stimulus, actuating the stimulus, and recording the human's response may be repeated for a given number of cycles. A record of the human's response to the stimuli may be used to update the distribution. For example, a change in the response with time for a given visual field location may be used to update the distribution. The distribution may be automatically updated. The subset of actuable elements may be a single actuable element. The actuable elements may be pixels of a computer display. The retinal area targeted by the subset of the elements may be in increasing relationship with corresponding distance from the center of the human's visual field. For example, larger subsets may be selected to target peripheral visual field regions and smaller subsets to target central visual field regions.

In another embodiment of the invention, there is a method for treating the visual system of the human comprising using a campimetric representation of the visual field to define at least a primary zone, a secondary zone, and a remainder zone. The remainder zone comprises that portion of the visual field that is outside of the other defined zones. A human is situated in proximity to a computer-actuated light emitting array that has a set of actuable elements. An actuable element subset is selected from the set of individually actuable elements and the subset is actuated to emit light stimulus directed to a specified region of the human's visual field. The selection of the actuable elements includes administering an apportioning bias in favor of the primary zone.

Various related embodiments are provided including optional or additional features. The steps of selecting an actuable element subset and actuating the elements to emit light stimulus may be repeated over a course of a therapeutic session so as to present a greater number of stimuli to the primary zone than to the secondary zone or to the remainder zone. The steps of selecting an actuable element subset and actuating the elements to emit light stimulus may be repeated over a course of a therapeutic session so as to present a greater number of stimuli to the primary zone than to the secondary zone or to the remainder zone, and a greater number of stimuli to the secondary zone than to the remainder zone. The number of stimuli presented to the remainder zone may be non-zero. A further step of recording the human's response to the stimuli may be included. A cycle of selecting a stimulus, actuating the stimulus and recording the human's response may be repeated for a given number of cycles. The record of the human's response to the stimuli may be used to redefined the primary zone or the secondary zone. A change in the response with time for a given visual field location may be used to redefine the zone. The redefinition may be done automatically. The subset of actuable elements may be a single actuable element. The retinal area targeted by the subset of elements may increase with corresponding distance from the center of the human's visual field. For example, larger subsets of elements may be selected to target peripheral visual field regions and smaller subsets selected to target central visual field regions.

In accordance with yet another embodiment of the invention, a method is provided for treating the visual system of a human. The method includes using a campimetric representation of the visual field to define a transition zone that is bordered by a blind zone and an intact zone. A human is situated in proximity to a computer-actuated light emitting array having a set of actuable elements. A subset of actuable elements is selected and actuated to a emit light stimulus directed to the transition zone of the human's visual field. The selecting and actuating steps are repeated to effectuate a course of therapy. The selection includes a bias for central visual field regions.

Various related embodiments are provided including optional or additional features. The method may include recording the human's response to the stimuli. The record of the human's response to the stimuli may be used to update the definition of the transition zone. Updating the transition zone may include using a change in the response with time for a given visual field location. The zone may be redefined automatically.

In a further embodiment of the invention, there is a system for treating the visual system of a patient. The system includes a display that has an array of individually actuable light emitting elements adapted to present stimuli to a human during a course of therapy. The system also includes an apportioner that is adapted to accept a campimetric representation of the visual field and apportion a sequence of stimuli to specified regions of the visual field. The system further includes an actuator for actuating display elements according to the apportionment of the apportioner. The apportioner apportions a greater share of stimuli to those the visual field regions nearer the center of the visual field.

Various related embodiments are provided including optional or additional features. The actuator may present a fixation stimulus to the human. The system may include software and/or hardware for recording the human's response to the stimuli, for using the record of the human's response to the stimuli to allocate future stimuli and/or for using a change in the response with time for a given visual field location to allocate future stimuli. The system may include software and/or hardware for varying the targeted retinal area of the presented stimulus so as to increase with corresponding distance from the center of the human's visual field. The retinal area may be targeted by the subset of elements sized in increasing relationship with corresponding distance from the center of the human's visual field by selecting larger stimuli to target peripheral visual field regions and smaller subsets to target central visual field regions.

In a further embodiment of the invention, there is a system for treating the visual system of a patient. The system includes a display having an array of individually actuable light emitting elements adapted to present stimuli to a human during a course of therapy and an apportioner that is adapted to accept a campimetric representation of the visual field and apportion a sequence of stimuli to specified regions with the visual field. The system also includes an actuator for actuating display elements according to the apportionment of the apportioner. The apportioner apportions by using a campimetric representation of the visual field to define at least a primary zone, a secondary zone, and a remainder zone, the remainder zone comprising that portion of the visual field that is outside of the other defined zones.

Various related embodiments are provided including optional or additional features. The system may include software and/or hardware for presenting a greater number of stimuli to the primary zone than to the secondary zone or to the remainder zone. The system may include software and/or hardware for presenting, over the course of therapy, a greater number of stimuli to the primary zone than to the secondary zone or to the remainder zone, and a greater number of stimuli to the secondary zone than to the remainder zone. The number of stimuli presented to the remainder zone may be non-zero. The system may include software and/or hardware for recording the human's response to the stimuli, for using the record of the human's response to the stimuli to allocate future stimuli and/or for using a change in the response with time for a given visual field location to allocate future stimuli. The system may include software and/or hardware for increasing the targeted retinal area of the presented stimulus increases with corresponding distance from the center of the human's visual field. The retinal area may be targeted by the subset of elements in increasing relationship with corresponding distance. The system may include software and/or hardware for using the record of the human's response to the stimuli to redefine one of the primary zone and the secondary zone. The system may use a change in the response with time for a given visual field location to redefine one of the primary zone and the secondary zone. The zone may be automatically redefined.

In a further embodiment of the invention, there is a system for treating the visual system of a patient. The system includes a display having an array of individually actuable light emitting elements adapted to present stimuli to a human during a course of therapy and an apportioner that is adapted to accept a campimetric representation of the visual field and apportion a sequence of stimuli to specified regions with the visual field. The system also includes an actuator for actuating display elements according to the apportionment of the apportioner. The apportioner apportions by using a campimetric representation of the visual field to define at a transition zone bordered by a blind zone and an intact zone and apportion stimuli to the transition zone with a bias toward the central visual field.

Various related embodiments are provided including optional or additional features. The system may include software and/or hardware for recording the human's response to the stimuli, for using the record of the human's response to the stimuli to update the transition zone, for updating the transition zone using a change in the response with time for a given visual field location, and for automatically redefining the transition zone.

### BRIEF DESCRIPTION OF THE DRAWINGS

The foregoing features of the invention will be more readily understood by reference to the following detailed description, taken with reference to the accompanying drawings, in which:

FIG. 1 shows a flow chart of a method of visual restoration therapy in accordance with an embodiment of the invention;

FIG. 2 shows an example of a training area having three (3) sub-areas;

FIG. 3 shows a visual field map with a blind zone, intact zone and transition zones;

FIG. 4 shows a visual field map with a defined border zone and central visual field;

FIG. 5 shows a visual field map with multiple defined apportionment zones;

FIG. 6 shows a visual field map before and after therapy;

FIG. 7 shows how a particular visual field location may improve with therapy.

### DETAILED DESCRIPTION OF SPECIFIC EMBODIMENTS

Definitions. As used in this description and the accompanying claims, the following terms shall have the meanings indicated, unless the context otherwise requires:
“VRT” shall mean visual restoration therapy, a therapeutic process for selectively targeting and stimulating specific visual field regions;
An “light emitting array having a set of individually actuable elements” shall mean any device capable of transmitting an illuminating pattern to the retina of a human including a cathode ray tube (CRT), liquid crystal display (LCD), organic light emitting diodes (OLED), or other such device which may be placed at various distances from a patient's eyes, and includes head mounted displays and image projection methods including the use of digital light processing (DLP™)
In the context of visual restoration therapy, a “campimetric representation” shall mean any data set that associates a set of visual field positions with at least one corresponding patient performance data set. The patient performance data may include patient response times, or threshold intensity required to observe a stimulus;
A “course of VRT” shall mean any temporally continuous or discontinuous VRT session, which may span minutes, hours, days, weeks, months, or years;
An “apportioner” shall mean any device including hardware, software or both capable of apportioning a finite number of individual light stimuli events between specified visual field zones according to a particular bias. For example, the apportioner may be embodied in a computer with software for administering VRT. In the context of an apportioner, a “bias” shall mean a skewing of stimulus apportioning so as to stimulate a particular zone to a greater degree than one or more other zones.
An “actuator” shall mean any device capable of switching array elements so as to illuminate the retina of a patient with a given pattern and distribution of stimuli

Aspects of the present invention may solve the problems outlined above by apportioning or rationing stimuli over a course of VRT so as to optimize stimulation to obtain more significant clinical outcomes when using limited amounts of light stimuli. For a given length of therapy (e.g., a single session, or a course of therapy over weeks or months), a patient will receive a finite number of stimuli. For example, a patient may receive 500-600 stimuli in a 20 to 30 minute VRT session. A therapist may desire to stimulate multiple visual field zones (e.g., both functionally important central areas and ARVs). However, a tradeoff must be made between the number of stimuli directed at a given zone and areal coverage. Illustrative embodiments of the present invention may solve some of these or other problems by dividing a therapy area into regions and applying different stimuli densities to each region. Unless otherwise indicated, the operations of the VRT systems described below may be fully automatic in the sense that the therapist need not intervene during a therapy session or even a multi-session course of therapy.

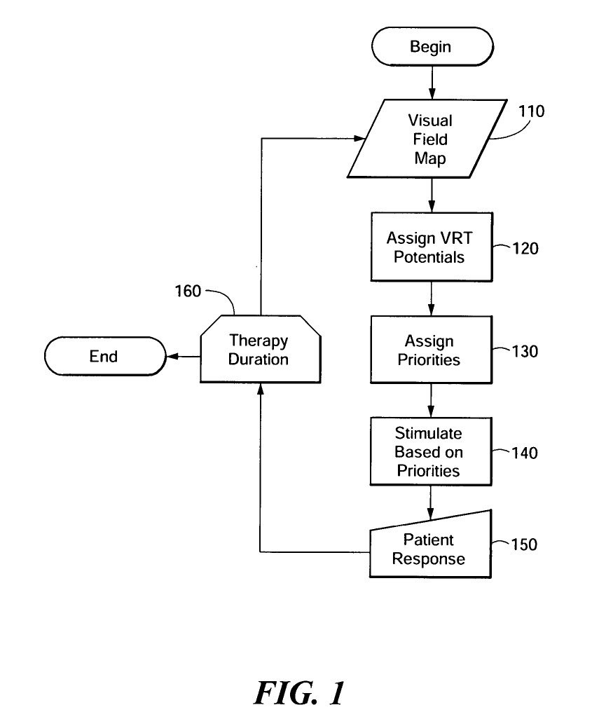

FIG. 1 shows a flow chart in accordance with an embodiment of the invention. The flow chart represents a method that may be embodied in a VRT apparatus or software module for use with a VRT apparatus. As is known in the art, a patient is situated in front of a display and instructed to fixate upon a fixation point or stimulus. The display may be a computer driven CRT, LCD, OLED, DLP, plasma display, or other such display including a head mounted display (e.g., goggles or helmet). The display has associated hardware for actuating subsets of individual elements of the display from a set (e.g. pixels or subsets of pixels) in order to target a specific area of the patient's retina and neuronal components of their visual field with a patterned illumination. The patterned illumination may be a single pixel, a contiguous subset of pixels that project a particular shape, or even a discontiguous subset of pixels. Targeting of the illumination pattern upon the retina may be accomplished by applying a specified offset from a point upon which the patient is instructed to fixate (i.e. a fixation point).

A campimetric representation of the patient's visual field (a “visual field map”) is obtained (step 110). The representation may be manifested as a multi-dimensional data set or visual field map, either as an array in computer memory, or expressed graphically. The campimetric representation may be the result of a previous VRT session or other campimetric activity. For example, the campimetric representation may contain, as a function of position relative to the a fixation point, or in an array corresponding to pixels on a VRT display, response times, fraction of correct responses, or other data related to the sensitivity of the patient's visual field neurons to light stimuli. Alternately, rather than starting with the map, the map can be generated through subsequent steps in the process, such a those listed below.

The campimetric representation is then used to assign the potential for a given neuron or visual field area to respond to VRT (step 120). For example, depending on the type of therapy chosen by the therapist, regions of the visual field that are partially responding, or are in a transition zone between a blind zone and an intact (i.e., seeing) zone may be indicative of a high recovery potential. Scores may be assigned based on potential. In an another example, a visual field location corresponding to a pixel element of a VRT may be assigned a low score if bounded on all sides by nonresponsive locations (i.e., blind regions), or bounded on all sides by intact regions, whereas locations bounded by both blind and intact locations, or one or more partially responding locations, may be awarded a higher scores. Trending data, i.e. improvements or decreases in patient responses in a given visual field areas may also be used to assign priorities; e.g., stimuli may be better invested in those areas showing an improvement with time. The result of step 120 may be used as a priority map, which may be used to distribute (i.e., apportion) stimuli among multiple locations.

In a specific example of a scoring system, points are awarded to each element in a two dimensional array of VRT pixel locations as follows:

i) locations adjacent to 8 blind locations (locations include diagonal locations)-0 points;

ii) locations adjacent to 8 intact locations—0 points;

iii) locations adjacent to one or more partially responding locations—1 point for each partially responding location;

iv) locations adjacent to both blind and intact locations—5 points

Optionally, additional factors may be used to assign priorities (step 130). Examples of additional factors include therapist intervention, or application of additional biases, which may be arrived at by using physiological or statistical factors. In one embodiment, a physiological bias is included that favors more central visual field regions over more peripheral regions so as to create a stimulus distribution that effects presentation of a larger fraction of the administered stimuli to more central regions. Thus, result may be desirable because more central regions of the visual field (e.g., the center 3-5°) have more neuronal synapses and are thus critical in certain key activities such as reading. The stimuli distribution can be tuned to match approximately the number of neuronal synapses at a given location (i.e. the cortical magnification factor) by using population-derived visual field structures, or maps of the individual patient's visual field.

Stimuli are apportioned to the patient based on the assigned priorities by actuating the individual actuable light-emitting elements of a display device to target a specified region of the patient's visual field. Various techniques are available to apportion the stimuli, including:

i) randomly assigning locations, and multiplying by a weighting factors based on a corresponding scores from those location obtained from the priority map; and

ii) populating a location table with a list of locations, the locations having a frequency that is proportional to priority scores. The sequence of location presentations may then be randomized. Additionally, the list may be further sorted to temporal clustering of stimuli presentations in a given area or zone.

After presenting a given stimuli, a patient response may be recorded; e.g., the by detection of a button actuation by the patient. Response times may also be recorded. Patient responses may be used to update the visual field map “in real time,” i.e., prior to completion of a therapy session or course of therapy. In other words, the loop is closed by returning to step 110 and repeating the loop for the duration of therapy (step 160). As discussed above, derivative aspects of the patient response, including temporal improvements of the patient response accuracy, response time, or threshold intensity required for a patient to see a stimulus may be utilized in setting priorities and assigning the apportioning distribution. Alternately, the loop may be closed by returning to step 140.

In accordance with another embodiment, the effectiveness of stimulus allocation is improved by varying the size of a stimulus according to the selected visual field location targeted by the stimulus. Because visual field resolution decreases with distance from the center of the visual field in a known way, stimulation of various neurons can be accomplished with different stimulus sizes (e.g., by altering the number of adjacent elements actuated). This approach may result in improved economies of stimulation allocation. For example, a computerized VRT apparatus may use an algorithm that randomly distributes stimuli, but avoids repetitive stimulation in the same location; using larger stimuli in peripheral regions of the visual field will result in a bias in favor of the central visual field. The larger stimuli are sized so as to illuminate a larger area of the patient's retina.

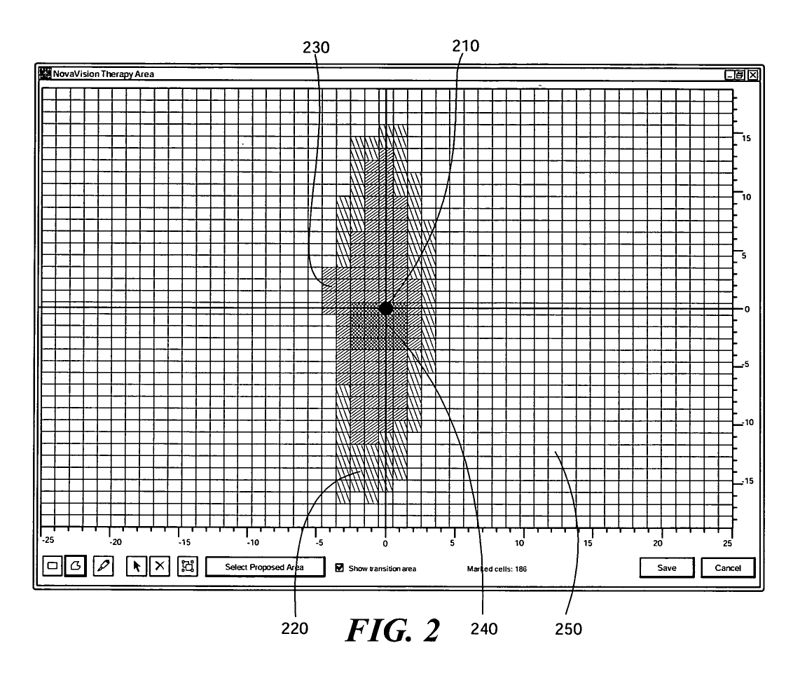

FIG. 2 shows a visual field map of a patient. Because the map would be generated using a VRT device, it is pixelated according to an array of actuable light emitting elements associated with a VRT apparatus. A fixation point 210 is used as a reference. The map has been divided into four zones: a primary zone 230, a secondary zone 220, a tertiary zone 240, and remainder zone 250 (the area not defined as one of the other zones). Although shown as continuous zones, the zones may also be discontinuous. The zones may be defined automatically or manually according to campimetric data. In an embodiment of the invention, a given, finite number of stimuli are apportioned among two or more zones. For example, 80% of the stimuli may be apportioned to the primary zone 230 and 20% to the secondary zone. Alternately, some fraction may be reserved for the tertiary zone, higher order zones, the remainder zone, or combinations thereof.

In a specific embodiment, the transition zone is defined as the primary zone and receives the majority of the stimuli, e.g., 70%. The remaining stimuli are presented to a border region secondary zone. The transition zone may be either continuous or discontinuous. The border region may be sized to extend beyond the transition zone into both blind and intact zones by a certain amount. For example, approximately two visual field cells on either side of the transition zone may be targeted. The patient response record may be used, periodically or continuously, to update the transition zone definition. A central visual field bias may also be applied to the transition zone.

In a related embodiment, vision is more intensely stimulated in a given zone, yet patient response is tracked by stimulating outside the given zone with fewer stimuli, and optionally, at a lower frequency (i.e., stimuli per unit time) in order to track patient response across a larger visual field region or the entire visual field. In this way, the various cells in the visual field are reconnoitered for potential recruitment into the set of locations receiving the more intense or frequent therapy. For example, in this way, a cell may be discovered to be exhibiting a recovery trend and the therapy regimen is adjusted accordingly (either automatically, or manually)

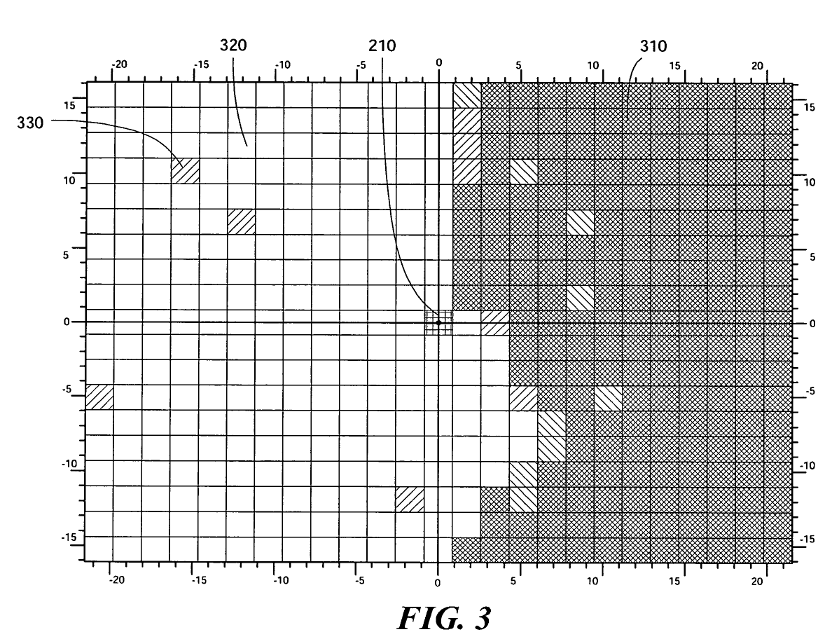

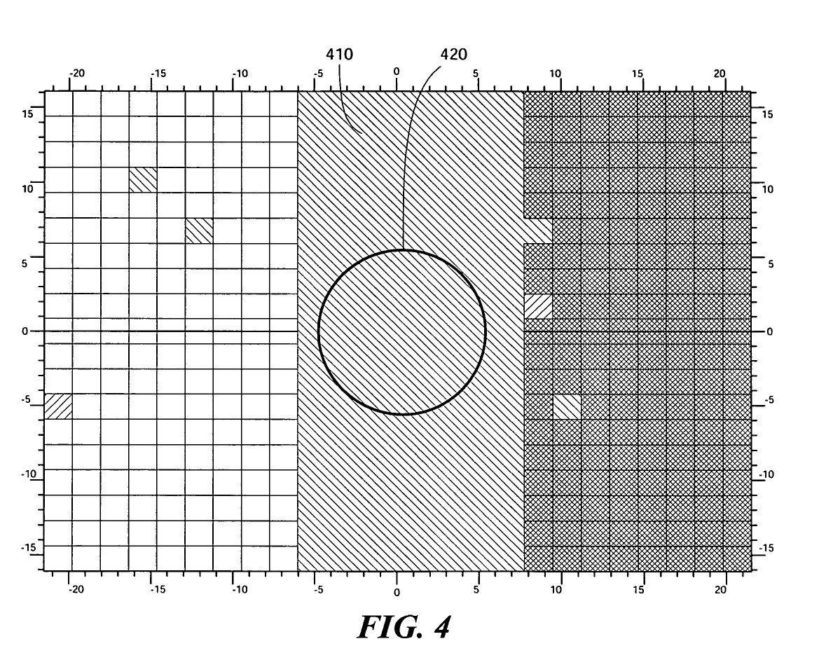

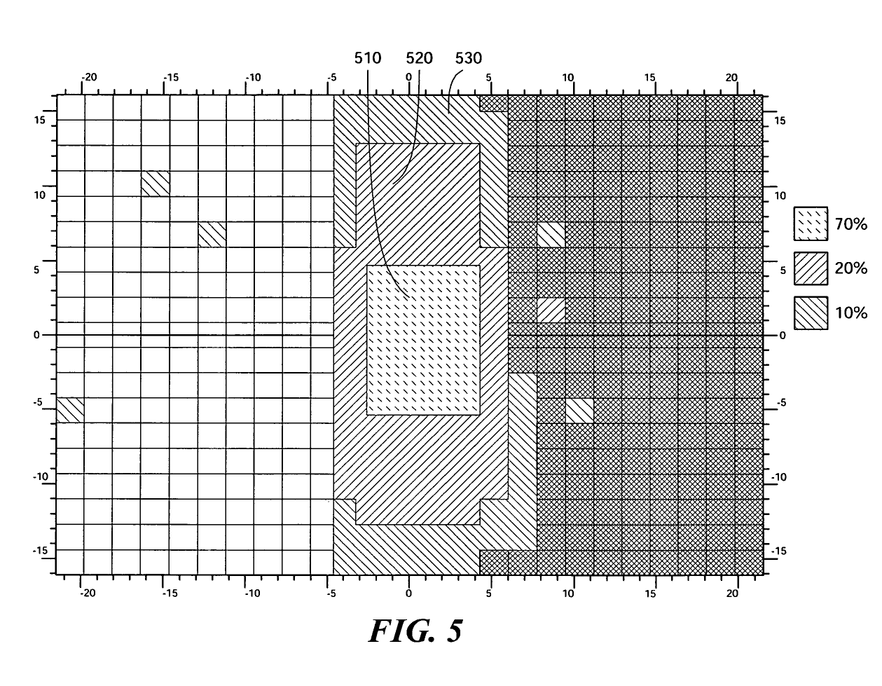

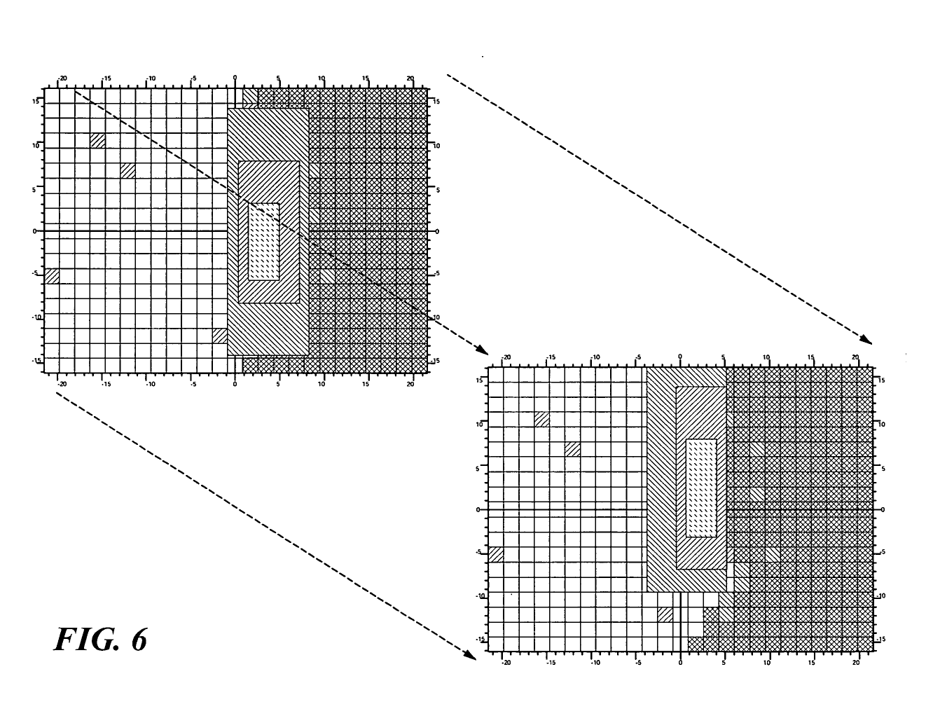

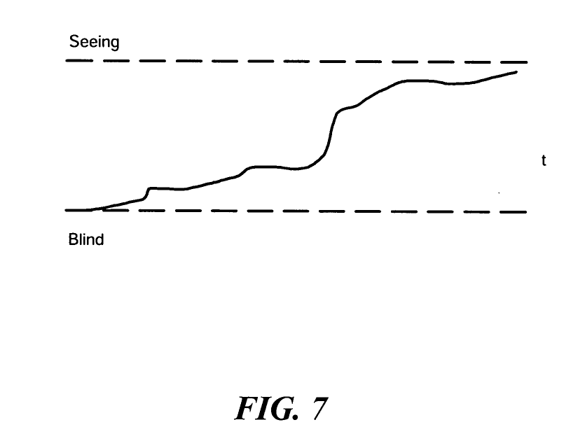

FIGS. 3-7 show a VRT process according to an embodiment of the present invention. FIG. 3 shows, in map format, a campimetric representation of a patient's visual field obtained using a VRT apparatus. A transition zone is defined by non-contiguous high-potential regions 330, portions of which lie within an intact zone 320, a blind zone 310, or adjacent both the intact and blind zones. FIG. 4 shows a border region 410 and a central field region 420 that may be used in assigning stimulus apportioning priorities. Accordingly, as shown in FIG. 5, multiple zones may be created. Thus, a primary zone 510 is apportioned 70% of the stimuli, a secondary zone 520 is apportioned 20% of the stimuli, and a noncontiguous tertiary zone 530 is apportioned 10% of the stimuli. FIG. 6 shows how, after a course of VRT treatment with the so-apportioned stimuli, the zones may be redefined to reflect changes in the patient's responsiveness (improvement or deterioration). FIG. 7 shows how a particular map element corresponding to a particular neuron may indicate improvement in responsiveness of the particular neuron over a course of stimulative therapy.

In various embodiments, zones may be defined and redefined automatically. However, tools may be provided to a therapist to define zones manually. For example, zones may be drawn on a computer screen so as to overlay a visual representation of the visual field (e.g., a campimetric map). For example, circles or ovals may be drawn to demark a zone for preferential stimulus apportionment.

In alternative embodiments, the disclosed methods for stimulative therapy may be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems.

Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some embodiments of the invention may be implemented as a combination of both software (e.g., a computer program product) and hardware. Still other embodiments of the invention are implemented as entirely hardware, or entirely software (e.g., a computer program product).

The described embodiments of the invention are intended to be merely exemplary and numerous variations and modifications will be apparent to those skilled in the art. All such variations and modifications are intended to be within the scope of the present invention as defined in the appended claims.

What is claimed is:

1. A method for treating the visual system of a human characterized by a visual field having a central portion, the method comprising:
(a) situating the human in proximity to a computer-actuated light emitting array having a set of individually actuable elements;
(b) using a campimetric representation of the visual field to select a stimulus distribution that is biased toward the central visual field;
(c) selecting an actuable element subset from the set of individually actuable elements based on the stimulus distribution; and
(d) actuating the subset of elements to emit a light stimulus directed to a specified region of the human's visual field;

2. A method according to claim 1, further comprising presenting a fixation stimulus to the human.

3. A method according to claim 1, further comprising repeating steps (c) and (d) for a given number of cycles.

4. A method according to claim 1, further comprising the step of (e) recording the human's response to the stimuli.

5. A method according to claim 4, further comprising repeating steps (c), (d) and (e) for a given number of cycles.

6. A method according to claim 5, further comprising using the record of the human's response to the stimuli to update the distribution.

7. A method according to claim 6, wherein using the record of the human's response to the stimuli to update the distribution further comprises using a change in the response with time for a given visual field location.

8. A method according to claim 6, wherein the distribution is updated automatically.

9. A method according to claim 1, wherein the subset comprises a single actuable element.

10. A method according to claim 1 wherein the individually actuable elements comprise pixels of a computer display.

11. A method according to claim 1, wherein the retinal area targeted by the subset of elements increases with corresponding distance from the center of the human's visual field.

12. A method according to claim 11, wherein the retinal area targeted by the subset of elements is in increasing relationship with corresponding distance from the center of the human's visual field by selecting larger subsets to target peripheral visual field regions and smaller subsets to target central visual field regions.

13. A method for treating the visual system of a human characterized by a visual field having a central portion, the method comprising:
(a) using a campimetric representation of the visual field to define at least a primary zone, a secondary zone, and a remainder zone, wherein the remainder zone comprises that portion of the visual field that is outside of the other defined zones;
(b) situating the human in proximity to a computer-actuated light emitting array having a set of actuable elements;
(c) selecting an actuable element subset from the set of individually actuable elements; and
(d) actuating the subset of elements to a emit light stimulus directed to a specified region of the human's visual field, wherein the selection of the actuable elements includes administering an apportioning bias in favor of the primary zone.

14. A method according to claim 13, further comprising repeating at least steps (c) and (d), over the course of a therapeutic session so as to present a greater number of stimuli to the primary zone than to the secondary zone or to the remainder zone.

15. A method according to claim 14, further comprising repeating at least steps (c) and (d), over the course of a therapeutic session so as to present a greater number of stimuli to the primary zone than to the secondary zone or to the remainder zone, and a greater number of stimuli to the secondary zone than to the remainder zone.

16. A method according to claim 15, wherein the number of stimuli presented to the remainder zone is non-zero.

17. A method according to claim 13, further comprising the step of (e) recording the human's response to the stimuli.

18. A method according to claim 17, further comprising repeating steps (c), (d) and (e) for a given number of cycles.

19. A method according to claim 17, further comprising using the record of the human's response to the stimuli to redefine one of the primary zone and the secondary zone.

20. A method according to claim 19, wherein using the record of the human's response to the stimuli to redefine further comprises using a change in the response with time for a given visual field location.

21. A method according to claim 19, wherein the zone is redefined automatically.

22. A method according to claim 17, wherein the subset comprises a single actuable element.

23. A method according to claim 13, wherein the retinal area targeted by the subset of elements increases with corresponding distance from the center of the human's visual field.

24. A method according to claim 23, wherein the retinal area targeted by the subset of elements is in increasing relationship with corresponding distance from the center of the human's visual field by selecting larger subsets to target peripheral visual field regions and smaller subsets to target central visual field regions.

25. A method for treating the visual system of a human characterized by a visual field having a central portion, the method comprising:
(a) using a campimetric representation of the visual field to define a transition zone bordered by a blind zone and an intact zone;
(b) situating the human in proximity to a computer-actuated light emitting array having a set of actuable elements;
(c) selecting an actuable element subset from the set of individually actuable elements;
(d) actuating the subset of elements to a emit light stimulus directed to the transition zone of the human's visual field; and
(e) repeating steps (c) and (d) to effectuate a course of therapy, wherein the selection includes a bias for central visual field regions.

26. A method according to claim 25, further comprising recording the human's response to the stimuli.

27. A method according to claim 26, further comprising using the record of the human's response to the stimuli to update the definition of the transition zone.

28. A method according to claim 27, wherein updating the definition of the transition zone includes using a change in the response with time for a given visual field location.

29. A method according to claim 27, wherein the zone definition is redefined automatically.

30. A system for treating the visual system of a patient, the system comprising:
a display having an array of individually actuable light emitting elements adapted to present stimuli to a human during a course of therapy;
an apportioner adapted to accept a campimetric representation of the visual field and apportion a sequence of stimuli to specified regions of the visual field; and
an actuator for actuating display elements according to the apportionment of the apportioner,
wherein the apportioner apportions a greater share of stimuli to those the visual field regions nearer the center of the visual field.

31. A system according to claim 30, wherein the actuator presents a fixation stimulus to the human.

32. A system according to claim 30, further comprising means for recording the human's response to the stimuli.

33. A system according to claim 32, further comprising means for using the record of the human's response to the stimuli to allocate future stimuli.

34. A system according to claim 33, further comprising means for using a change in the response with time for a given visual field location to allocate future stimuli.

35. A system according to claim 30, further comprising means for increasing the targeted retinal area of the presented stimulus increases with corresponding distance from the center of the human's visual field.

36. A system according to claim 35, wherein the retinal area targeted by the subset of elements is in increasing relationship with corresponding distance from the center of the human's visual field by selecting larger stimuli to target peripheral visual field regions and smaller subsets to target central visual field regions.

37. A system for treating the visual system of a patient, the system comprising:
a display having an array of individually actuable light emitting elements adapted to present stimuli to a human during a course of therapy;
an apportioner adapted to accept a campimetric representation of the visual field and apportion a sequence of stimuli to specified regions with the visual field; and
an actuator for actuating display elements according to the apportionment of the apportioner,
wherein the apportioner apportions by using a campimetric representation of the visual field to define at least a primary zone, a secondary zone, and a remainder zone, the remainder zone comprising that portion of the visual field that is outside of the other defined zones.

38. A system according to claim 37, further comprising means for presenting a greater number of stimuli to the primary zone than to the secondary zone or to the remainder zone.

39. A system according to claim 3730, further comprising means for presenting, over the course of therapy, a greater number of stimuli to the primary zone than to the secondary zone or to the remainder zone, and a greater number of stimuli to the secondary zone than to the remainder zone.

40. A system according to claim 37, wherein the number of stimuli presented to the remainder zone is non-zero.

41. A system according to claim 37, further comprising means for recording the human's response to the stimuli.

42. A system according to claim 41, further comprising means for using the record of the human's response to the stimuli to redefine one of the primary zone and the secondary zone.

43. A system according to claim 42, further comprising using a change in the response with time for a given visual field location to redefine one of the primary zone and the secondary zone.

44. A system according to claim 37, further comprising means for automatically redefining the zone.

45. A system according to claim 37, further comprising means for increasing the retinal area targeted by the stimulus with corresponding distance from the center of the human's visual field.

46. A system according to claim 45, further comprising means for selecting larger stimuli to target peripheral visual field regions and smaller stimuli to target central visual field regions.

47. A system for treating the visual system of a patient, the system comprising:
a display having an array of individually actuable light emitting elements adapted to present stimuli to a human during a course of therapy;
an apportioner adapted to accept a campimetric representation of the visual field and apportion a sequence of stimuli to specified regions with the visual field; and
an actuator for actuating display elements according to the apportionment of the apportioner,
wherein the apportioner uses a campimetric representation of the visual field to define at a transition zone bordered by a blind zone and an intact zone and apportion stimuli to the transition zone with a bias toward the central visual field.

48. A system according to claim 47, further comprising means for recording the human's response to the stimuli.

49. A system according to claim 48, further comprising means for using the record of the human's response to the stimuli to update the transition zone.

50. A system according to claim 49, further comprising means for updating the transition zone using a change in the response with time for a given visual field location.

51. A system according to claim 49, further comprising means for automatically redefining the transition zone.
